(19) United States
(12) Patent Application Publication
(10) Pub. No.: US 2018/0308567 A1
(43) Pub. Date: Oct. 25, 2018

(54) SYSTEM AND METHOD FOR STORING AND DELIVERING HEALTHCARE INFORMATICS DATA

(71) Applicants: RANDEEP SINGH, Pitampur (IN); SHAILESH PRITHANI, Jaipur (IN)

(72) Inventors: RANDEEP SINGH, Pitampur (IN); SHAILESH PRITHANI, Jaipur (IN)

(21) Appl. No.: 15/741,747

(22) PCT Filed: Jan. 6, 2017
(86) PCT No.: PCT/IN2017/050008

§ 371 Date: Jan. 3, 2018

(30) Foreign Application Priority Data
Jan. 6, 2016 (IN) 3630/DEL/2015

(51) Int. Cl. G16H 10/65; G16H 80/00

(52) U.S. Cl. CPC G16H 10/65; G06F 17/30312; G16H 80/00

(57) ABSTRACT

The embodiments herein provide a system for delivering healthcare informatics data that puts patients at the center of care and in control of the data generated about their particular medical condition. The system is intuitive and easy to use for clinicians, nurses, pharmacists, hospitals and diagnostics tests. The system includes a doctor who prints a prescription containing a unique PageID upon seeing a patient and stores the same in a database at a server. The PageID is linked to PatientID and other patient particulars so that the corresponding prescription is easily shareable and searchable in the database. A virtual printer driver is run on a computing device of the doctor/clinician to share the digital data with the backend server to generate a QR code or Barcode to be printed on the prescription paper. Anyone with relevant permissions accesses the original printed data by accessing a corresponding code.

### CROSS-REFERENCE TO RELATED APPLICATIONS

The embodiments herein claims the priority of the Indian Provisional Patent Application (PPA) filed on Nov. 6, 2015 and subsequently postdated by 2 months to Jan. 6, 2016 with the number 3630/DEL/2015 and entitled, “SYSTEM AND METHOD FOR DELIVERING HEALTHCARE INFORMATICS DATA”, and the contents of which are included in entirety as reference herein. The present application is a national phase application of the PCT application PCT/IN20171050008 filed on Jan. 6, 2017.

### BACKGROUND

### Technical Field

The embodiments herein are generally related to a healthcare informatics data management. The embodiments herein are particularly related to a system and method for storing and delivering healthcare informatics data. The embodiments herein are more particularly related to a system and method for a controlled access, distribution, delivery and consumption of healthcare informatics data. The embodiments herein are especially related to a system and method for digitizing printed and handwritten healthcare information and contextually delivering the digitized information.

### Description of the Related Art

Healthcare data management is a very important aspect of managing and caring for the personal health of an individual. Over a course of time, an individual is susceptible to fall sick for a plurality of times and it is vital for the individual to manage and store the details of the history of illnesses, diseases and treatment. However, it is often a challenge to store the healthcare information in an easily accessible manner and in a safe location.

Clinicians and doctors usually type, write or speak-out the prescriptions and treatment methodologies for a patient. While the spoken information is mostly for the immediate benefit and information of the patient, the written or typed medical records are kept for long-term storage and future references. However, the medical records remain with clinicians or hospitals and the patients do not have an access to the original records always. In times of emergency or a change in treatment plan, the records are to be shared between different health care providers such as doctors or hospitals for effective treatment. The sharing of information between the various health care providers is not easy, as the information is not readily accessible and not available to the patient.

The patients sometimes request the hospital or doctor to share a physical copy or the digitized copy of the healthcare records. However, it is very difficult for a patient to store all the healthcare information in an integrated and methodical manner. Since there is no uniformity in the quality or the type of healthcare information shared by a hospital or a doctor, the healthcare information of an individual are often stored in a plurality of locations. This fragmented and often incomplete storage of information results in a lack of proper information about an individual's medical history.

Hence, there is a need for storing and delivering healthcare informatics data in an integrated and uniform manner. There is also a need for digitizing printed and handwritten healthcare information to provide an integrated approach to storage and delivery of healthcare information.

The abovementioned shortcomings, disadvantages and problems are addressed herein and which will be understood by reading and studying the following specification.

### OBJECT OF THE EMBODIMENTS HEREIN

The primary object of the embodiments herein is to provide a system and method for storing and delivering healthcare informatics data.

Another object of the embodiments herein is to provide a system and method for controlled access, distribution, delivery and consumption of healthcare informatics data.

Yet another object of the embodiments herein is to provide a system and method for digitizing printed and handwritten healthcare information, such as prescription and patient case history, d contextually delivering the digitized information.

Yet another object, of the embodiments herein is to provide a healthcare informatics system that puts the patients at the center of healthcare and in control of the data generated about their particular medical condition.

Yet another object of the embodiments herein is to provide a healthcare informatics system that is very intuitive and easy to use for healthcare providers and relevant stakeholders such as clinicians, nurses, pharmacists, hospitals and diagnostics tests providers.

Yet another object of the embodiments herein is to provide a healthcare informatics system to perform a data transfer from pen to device such as mobile, tablet, computer etc., through an online or offline process or streaming in real time or near real time.

Yet another object of the embodiments herein is to provide a healthcare informatics system to perform a data transfer from device such as mobile, tablet, computer etc., to cloud and back through an online or offline process or streaming in real time or near real time.

These and other objects and advantages of the embodiments herein will become readily apparent from the following summary and the detailed description taken in conjunction with the accompanying drawings.

### SUMMARY

The following details present a simplified summary of the embodiments herein to provide a basic understanding of the several aspects of the embodiments herein. This summary is not an extensive overview of the embodiments herein. It is not is to identify key/critical elements of the embodiments herein or to delineate the scope of the embodiments herein. Its sole purpose is to present the concepts of the embodiments herein in a simplified form as a prelude to the more detailed description that is presented later.

The other objects and advantages of the embodiments herein will become readily apparent from the following description taken in conjunction with the accompanying drawings.

The various embodiments herein provide a system and method for storing and delivering healthcare informatics data.

According to an embodiment herein, digital pens and paper are provided to enable digitizing patient information in a healthcare informatics system. Digital pens are almost as discreet as a normal ballpoint pen; they use real ink and they write on real (dotted) paper. The only difference is digitization of every pen stroke with high fidelity. Apart from normal note taking, they have been used in scientific use cases. Few clinical use cases include prescription writing and recording, patient journals, forms and open ended questions, field notes and transcription, graphic documentation (i.e. pressure sores and skin lesions, fundal height, anatomical position, tumor staging, etc.).

According to an embodiment herein, a system for storing and delivering healthcare informatics data is provided. The system comprises a hardware processor, a memory, a storage database, a digital writing apparatus, a tagged writing surface and a user-device. The tagged writing surface is a prescription sheet provided or embedded with tags or codes. The prescription sheet comprises pre-set, customized static information, and dynamic information. The pre-set, customized static information comprises hospital information, doctor information and tag information. The dynamic information comprises patient's name, patient's age, patient's disease information and suggested treatment for the diseases. The digital writing apparatus is configured to write dynamic information on the prescription sheet. The tag on the prescription sheet is a unique identification code or RFID code or QR code for a patient. The tag is provided to retrieve and digitize information written on the prescription sheet. The user-device is a computing device.

According to an embodiment herein, the digital writing apparatus is a writing instrument that comprises an imaging device and a communication module. The imaging device is configured to capture and convert any data physically written using the writing instrument into digital data. The digital writing apparatus is configured to transmit the digital data to a server or a remote server or cloud server through the communication module for storage and retrieval of the digitized information. The transmitted digital data are stored in the storage database provided in the server or remote server or cloud server. The communication module is selected from a group consisting of a wired network, wireless network, short range communication, Bluetooth communication and near field communication device.

According to an embodiment herein, the digital writing apparatus further comprises a memory for storing the captured and converted digital data.

According to an embodiment herein, the computing device is configured to connect to the storage database and retrieve healthcare informatics data related to a plurality of user accounts. The user accounts are created and configured in the computing device.

According to an embodiment herein, the storage database is configured to connect to the computing device and deliver contextual healthcare informatics data pertaining to a plurality of user accounts configured to the user device.

According to an embodiment herein, the computing device is selected from a group consisting of a PC, laptop, palm computer, mini-computer, portable computing device, computing tablet, iPad, iPod, and smart phone.

According to an embodiment herein, the system is configured to provide a healthcare informatics system to perform a data transfer from pen to device such as mobile, tablet, computer etc., through an online car offline process or streaming in real time or near real time.

According to an embodiment herein, the system is configured to provide a healthcare informatics system to perform a data transfer from device such as mobile, tablet, computer etc., to cloud and back through an online or offline process or streaming in real time or near real time.

According to an embodiment herein, a computer implemented method comprising instructions stored on a non-transitory computer readable, storage medium and executed on a computing device o provided with a hardware processor and a memory for storing and delivering healthcare informatics data. The method comprises the steps of: creating a prescription sheet comprising preset customized static information, dynamic information and a tag, where the tag is associated/configured with a unique digital identity, and wherein the tag is associated/configured with a unique digital identity, and wherein the preset customized static information comprises hospital information, doctor information and tag information, and wherein the dynamic information comprises patient's name, patient's age, patient's disease information and suggested treatment for the diseases, wherein the digital writing apparatus is configured to write dynamic information on the prescription sheet, and wherein the tag on the prescription sheet is a unique identification code or RFID code or QR code for a patient, and wherein the tag is provided to retrieve and digitize information written on the prescription sheet; writing dynamic information on the prescription sheet with a digital writing apparatus, where the digital writing apparatus is a writing instrument, and wherein the writing instrument comprises an imaging device and a memory, and wherein the imaging device is configured to capture and convert any data physically written on the prescription sheet with the writing instrument into a digital data, and the digital writing apparatus is configured to store the digitized information in the memory; transmitting the digital data to a server or a remote server or cloud server through a communication module provided in the digital writing apparatus for storage and retrieval of the digitized information, where the transmitted digital data are stored in the storage database provided in the server or remote server or cloud server, and where the communication module is selected from a group consisting of a wired network, wireless network, short range communication Bluetooth communication and near field communication device; storing the dynamic information written on the prescription sheet in the storage database; tagging the information stored in the storage database with the same tagging information comprised in the prescription sheet; and, delivering healthcare informatics data related to a particular tag to a plurality of user devices, and wherein the plurality of user devices are configured to receive the healthcare informatics data.

According to an embodiment herein, a method is provided for connecting the computing device to the storage database to retrieve healthcare informatics data related to a plurality of user accounts. The user accounts are created and configured in the computing device.

According to an embodiment herein, a method is provided for connecting the storage database to the computing device to deliver contextual healthcare informatics data related to a plurality of user accounts configured to the user device.

According to an embodiment herein, a method further comprises providing a healthcare informatics system to perform a data transfer from pen to device such as mobile, tablet, computer etc., through an online or offline process or streaming in real time or near real time.

According to an embodiment herein, a method further comprise providing a healthcare informatics system to perform a data transfer from device such as mobile, tablet, computer etc., to cloud and back through an online or offline process or streaming in real time or near real time.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof and the embodiments herein include all such modifications.

### BRIEF DESCRIPTION OF THE DRAWINGS

The other objects, features and advantages will occur to those skilled in the art from the following description of the preferred embodiment and the accompanying drawings in which:

FIG. 1 illustrates a block diagram of a healthcare information management system, according to an embodiment herein.

FIG. 2 illustrates a plan view of a sample (specimen) prescription paper with a pre-printed barcode at the top, according to one embodiment herein.

FIG. 3 illustrates a plan view of sample (specimen) prescription paper with a pre-printed QR code at bottom, according to one embodiment herein.

FIG. 4 illustrates a block diagram of an online platform/portal for data sharing and consumption in a healthcare information management system according to one embodiment herein.

FIG. 5 illustrates a system for data sharing and consumption in a healthcare information management system using digital pen and paper, according to one embodiment herein.

FIG. 6 illustrates a flow chart explaining a process for data sharing and consumption in a healthcare information management system using digital pen and paper, according to one embodiment herein.

Although the specific features of the embodiments herein are shown in some drawings and not in others. This is done for convenience only as each feature may be combined with any or all of the other features in accordance with the embodiment herein.

### DETAILED DESCRIPTION OF THE EMBODIMENTS HEREIN

In the following detailed description, a reference is made to the accompanying drawings that form a part hereof, and in which the specific embodiments that may be practiced is shown by way of illustration. These embodiments are described in sufficient detail to enable those skilled in the art to practice the embodiments and it is to be understood that the logical, mechanical and other changes may be made without departing from the scope of the embodiments. The following detailed description is therefore not to be taken in a limiting sense.

The various embodiments herein provide a system and method for storing and delivering, healthcare informatics data.

According to an embodiment herein, a system for storing and delivering healthcare it data is provided. The system comprises a hardware processor, a memory, a storage database, a digital writing apparatus, a tagged writing surface and a user-device. The tagged writing surface is a prescription sheet provided or embedded with tags or codes. The prescription sheet comprises pre-set, customized static information, and dynamic information. The pre-set, customized static information comprises hospital information, doctor information and tag information. The dynamic information comprises patient's name, patient's age, patient's disease information and suggested treatment for the diseases. The digital writing apparatus is configured to write dynamic information on the prescription sheet. The tag on the prescription sheet is a unique identification code or RFID code or QR code for a patient. The tag is provided to retrieve and digitize information written on the prescription sheet. The user-device is a computing device.

According to an embodiment herein, the digital writing apparatus is a writing instrument that comprises an imaging device and a communication module. The imaging device is configured to capture and convert any data physically written using the writing instrument into digital data. The digital writing apparatus is configured to transmit the digital data to a server or a remote server or cloud server through the communication module for storage and retrieval of the digitized information. The transmitted digital data are stored in the storage database provided in the server or remote server or cloud server. The communication module is selected from a group consisting of a wired network, wireless network, short range communication, Bluetooth communication and near field communication device.

According to an embodiment herein, the digital writing apparatus further comprises a memory for storing the captured and converted digital data.

According to an embodiment herein, the computing device is configured to connect to the storage database and retrieve healthcare informatics data related to a plurality of user accounts. The user accounts are created and configured in the computing device.

According to an embodiment herein, the storage database is configured to connect to the computing device and deliver contextual healthcare informatics data pertaining to a plurality of user accounts configured to the user device.

According to an embodiment herein, the computing device is selected from a group consisting of a PC, laptop, palm computer, mini-computer, portable computing device, computing tablet, iPad, iPod, and smart phone.

According to an embodiment herein, the system is configured to provide a healthcare informatics system to perform a data transfer from pen to device such as mobile, tablet, computer etc., through an online or offline process or streaming in real time or near real time.

According to an embodiment herein, the system is configured to provide a healthcare informatics system to perform a data transfer from device such as mobile, tablet, computer etc., to cloud and back through an online or offline process or streaming in real time or near real time.

According to an embodiment herein, a computer implemented method comprising instructions stored on a non-transitory computer readable storage medium and executed on a computing device is provided with a hardware processor and a memory for storing and delivering healthcare informatics data. The method comprises the steps of: creating a prescription sheet comprising preset customized static information, dynamic information and a tag, where the tag is associated/configured with a unique digital identity, and wherein the preset customized static information comprises hospital information, doctor information and tag information, and wherein the dynamic information comprises patient's name, patient's age, patient's disease information and suggested treatment for the diseases, wherein the digital writing apparatus is configured to write dynamic information on the prescription sheet, and wherein the tag on the prescription sheet is a unique identification code or RFID code or QR code for a patient, and wherein the tag is provided to retrieve and digitize information written on the prescription sheet; writing dynamic information on the prescription sheet with a digital writing apparatus, where the digital writing apparatus is a writing instrument, and wherein the writing instrument comprises an imaging device and a memory, and wherein the imaging device is configured to capture and convert any data physically written on the prescription sheet with the writing instrument, into a digital data, and the digital writing apparatus is configured to store the digitized information in the memory; transmitting the digital data to a server or a remote server or cloud server through a communication module provided in the digital writing apparatus for storage and retrieval of the digitized information, where the transmitted digital data are stored in the storage database provided in the server or remote server or cloud server, and where the communication module is selected from a group consisting of a wired network, wireless network short range communication Bluetooth communication and near field communication device; storing the dynamic information written on the prescription sheet in the storage database; tagging the information stored in the storage database with the same tagging information comprised in the prescription sheet; and, delivering healthcare informatics data related to a particular tag to a plurality of user devices, and wherein the plurality of user devices are configured to receive the healthcare informatics data.

According to an embodiment herein, a method is provided for connecting the computing device to the storage database to retrieve healthcare informatics data related to a plurality of user accounts. The user accounts are created and configured in the computing device.

According to an embodiment herein, a method is provided for connecting the storage database to the computing device to deliver contextual healthcare informatics data related to a plurality of user accounts configured to the user device.

According to an embodiment herein, a method further comprises providing a healthcare informatics system to perform a data transfer from pen to device such as mobile, tablet, computer etc., through an online or offline process or streaming in real time or near real time.

According to an embodiment herein, a method further comprise providing a healthcare informatics system to perform a data transfer from device such as mobile, tablet, computer etc., to cloud and back through an online or offline process or streaming in real time or near real time.

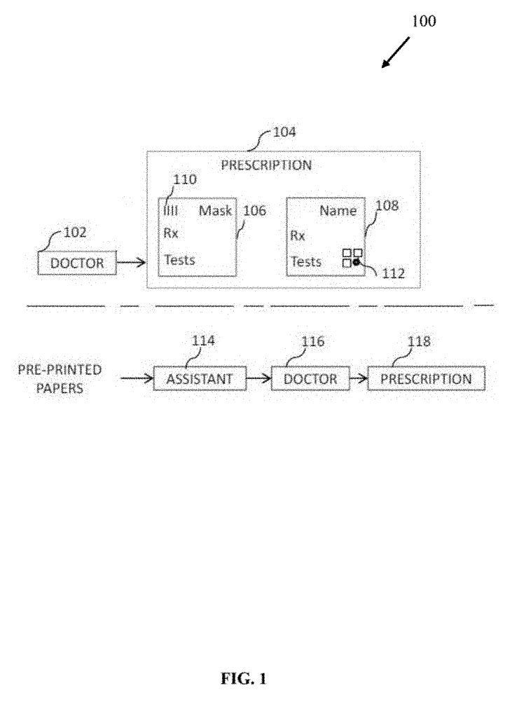

FIG. 1 illustrates a block diagram of a healthcare information management system, according to an embodiment herein. FIG. 1 illustrates an environment, where the embodiments herein are practiced. The environment 100 comprises a doctor 102, who prints a prescription 104 of a first type 106, or a second type 108 upon seeing a patient, and stores the same in a database at a backend server (not illustrated in the figure). The first type 106 of prescription includes a unique PageID, such as bar code 110, and the second type 108 of prescription includes a unique PageID such as QR code 112. In one embodiment, the PageIDs such as bar code 110 and QR code 112 are linked to PatientID and other patient particulars so that the corresponding prescription is easily shareable and searchable in the database at the backend server. In one embodiment herein, the clinician/doctor 102 possesses an electronic method of data entry such as HIS/EMR/EHR etc. and usually print out their prescriptions (or rarely case history) to be handed over to the patients. In this scenario, a virtual printer driver runs on a computing device of the doctor/clinician that may accomplish a) sharing of the digital (to be printed) data with a backend server, b) generating a QR code or Barcode and print on the (prescription) paper. Later on anyone with relevant permissions can click on the QR code 112 or Barcode 110 to net access to the original printed data. Note here that anything that clinician 102 writes with a pen may not be available to the accessing patient. In another embodiment herein, pre-printed digital papers are used by assistants 114 for filling the clinician prescription in smaller clinics and hospitals.

According to an embodiment herein, the system is configured to provide a healthcare informatics system to perform a data transfer from pen to device such as mobile, tablet, computer etc., through an online or offline process or streaming in real time or near real time.

According to an embodiment herein, the system is configured to provide a healthcare informatics system to perform a data transfer from device such as mobile, tablet, computer etc., to cloud and back through an online or offline process or streaming in real time or near real time.

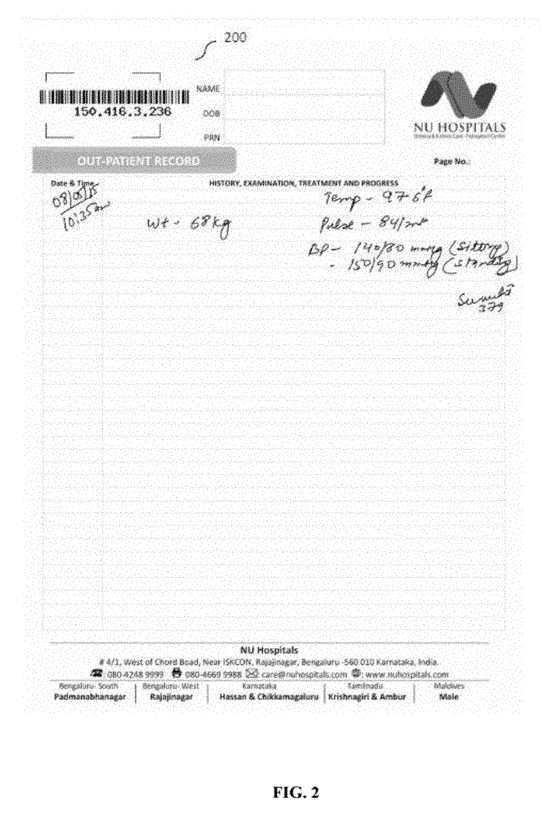

FIG. 2 illustrates a plan view of a sample (specimen) prescription paper with a pre-printed barcode at the top, according to one embodiment herein. Each sample prescription paper 200 is preprinted with a barcode that has a unique PageID. While generating a clinic specific barcode, inputs from the PageID are also input with the barcode to assist in clinical workflow. The data comprises inputs such as PRN (Patient Registration No.), Name, Age, Contact Details and Medical History information to generate a unique bar code.

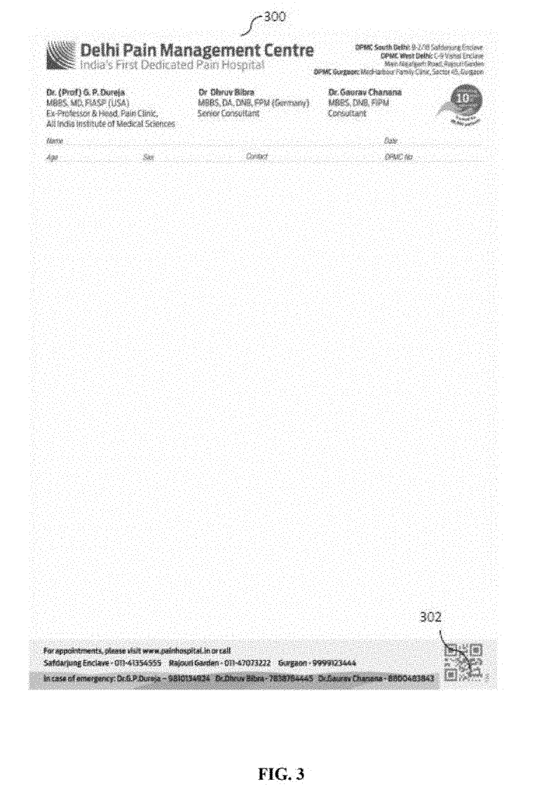

FIG. 3 illustrates a plan view of sample (specimen) prescription paper with a pre-printed QR code at bottom, according to one embodiment herein. Each digital paper in this case is pre-printed with a customized patient particular bar on top of the paper 300. The fields such as Name, Age and Sex are transcribed and tagged with a particular PageID. In this scenario, the digital pen provides the handwritten data (ink) on the paper for integration with the backend server. At a later stage the barcode/QRcode is scanned by a relevant entity to get access to the original data. Digital pens and papers provide a unique PageID and handwritten data (Ink).

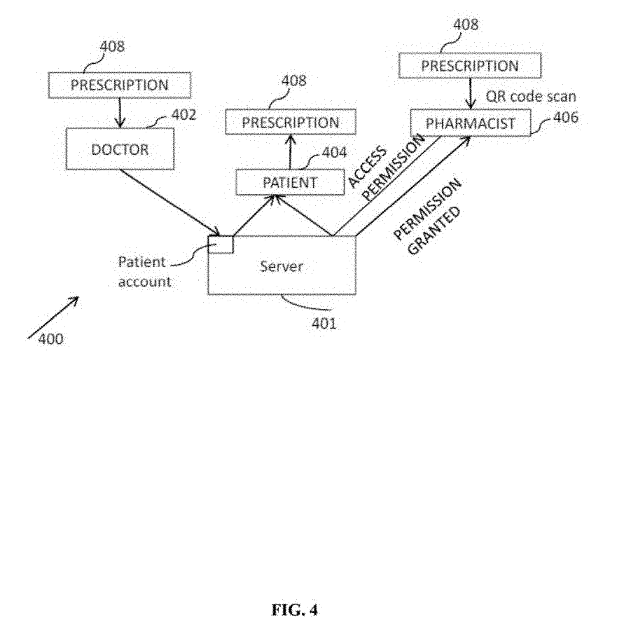

FIG. 4 illustrates a block diagram of an online platform/portal for data sharing and consumption in a healthcare information management system, according to one embodiment herein. The online platform 400 is hosted on a cloud and care providers such as clinicians, pharmacists, hospitals and diagnostics tests providers register for relevant patient data access. The online platform 400 includes a server 401 that provides a healthcare mobile application for execution at the computing devices of the doctor 402, patient 484, and pharmacist 406. Examples of the computing devices, include, but are not limited to, smart phones and tablets. In one embodiment, a doctor/clinician 402 may see a patient 404 at a clinic or hospital, and give the hard copy of the prescription 408 to the patient. The prescription (both preprinted and handwritten) may be saved at the server 401 and can be shared among the doctor 402, patient 404 and pharmacist 406 via mobile application or other online access means. In another embodiment, a clinician 402 consulting a patient 404 via phone or online methods may keep the hard copy of the prescription 408 with himself, and provide a soft copy of the prescription 408 (also known as tele-prescription) to the patient 404 via corresponding, mobile application. In yet another embodiment, if any other entity requires access to the original data, a request first reaches the server 401 where a count is maintained on who (clinician, pharmacists, hospitals or diagnostics provider) is accessing the data. An access permission may be sought from the patient 404 and then the soft copy may be served by the server 401. Thus, the access is controlled at the server 401 about who can access based on their clinical needs and profiles. In yet another embodiment, the server 401 may add value to the process by ‘tagging’ the forms and performing medical transcription so that when documents appear on the doctor's and patient's computing devices, they are easily searched.

According to an embodiment herein, the system is configured to provide a healthcare informatics system to perform a data transfer from pen to device such as mobile, tablet, computer etc., through an online or offline process or streaming in real time or near real time.

According, to an embodiment herein, the system is configured to provide a healthcare informatics system to perform a data transfer from device such as mobile, tablet, computer etc., to cloud and back through an online or offline process or streaming in real time or near real time.

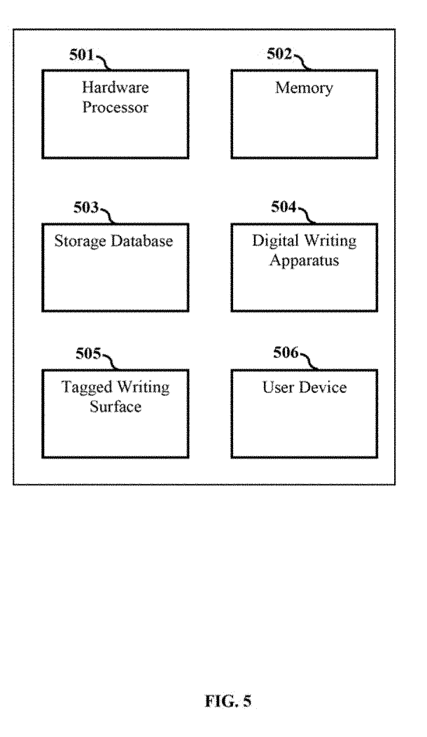

FIG. 5 illustrates a system for data sharing and consumption in a healthcare information management system using digital pen and paper, according to one embodiment herein. The system comprises a hardware processor 501, a memory 502, a storage database 503, a digital writing apparatus 504, a tagged writing surface 505 and a user-device 506.

According to an embodiment herein, the system is configured to provide a healthcare informatics system to perform a data transfer from pen to device such as mobile, tablet, computer etc., through an online or offline process or streaming in real time or near real time.

According to an embodiment herein, the system is configured to provide a healthcare informatics system to perform a data transfer from device such as mobile, tablet, computer etc., to cloud and back through an online or offline process or streaming in real time or near real time.

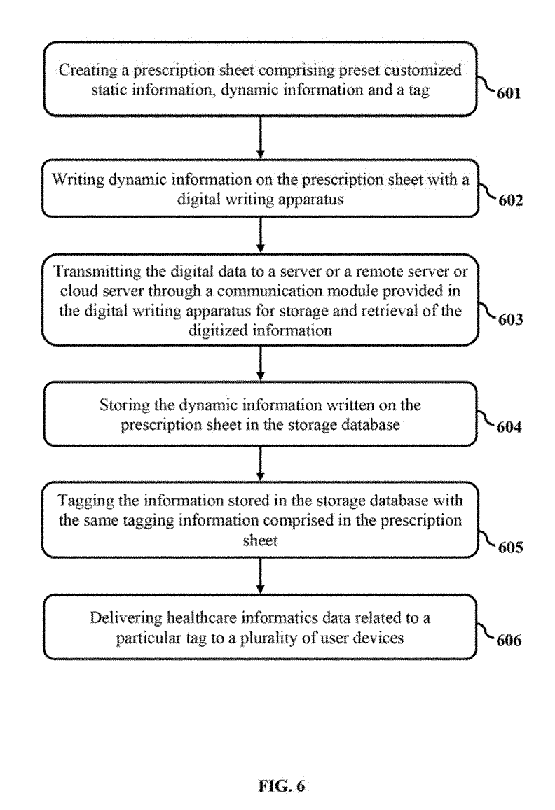

FIG. 6 Illustrates a flow chart explaining a process for data sharing and consumption in a healthcare information management system using digital pen and paper, according to one embodiment herein. The process comprises the following steps: creating a prescription sheet comprising preset customized static information, dynamic information and a tag (601); writing dynamic information on the prescription sheet with a digital writing apparatus (602); transmitting the digital data to a server or a remote server or cloud server through a communication module provided in the digital writing apparatus for storage and retrieval of the digitized information (603): storing the dynamic information written on the prescription sheet in the storage database (604); tagging the information stored in the storage database with the same tagging information comprised in the prescription sheet (605); and, delivering healthcare informatics data related to a particular tag to a plurality of user devices (606).

According to an embodiment herein, a method further comprises providing a healthcare informatics system to perform a data transfer from pen to device such as mobile, tablet, computer etc., through an online or offline process or streaming in real time or near real time.

According to an embodiment herein, a method further comprise providing a healthcare informatics system, to perform a data transfer from device such as mobile, tablet, computer etc., to cloud and back through an online or offline process or streaming in real time or near real time.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

Although the embodiments herein are described with various specific embodiments, it will be obvious for a person skilled in the art to practice the disclosure with modifications. However, all such modifications are deemed to be within the scope of the appended claims.

It is also to be understood that the following claims are intended to cover all of the generic and specific features of the embodiments described herein and all the statements of the scope of the embodiments which as a matter of language might be said to fall there between.

What is claimed is:

1. A system for storing and delivering healthcare informatics data, the system comprising:
a hardware processor;
a memory;
a storage database;
a digital writing apparatus;
a tagged writing surface, wherein the tagged writing surface is a prescription sheet provided or embedded with tags or codes; and,
a user-device, wherein the user-device is a computing device.

2. The system according to claim 1, wherein the digital writing apparatus is a writing instrument that comprises an imaging device and a communication module, and wherein the imaging device is configured to capture and convert any data physically written using the writing instrument into digital data, and wherein the digital writing apparatus is configured to transmit the digital data to a server or a remote server or cloud server through the communication module for storage and retrieval of the digitized information, and wherein the transmitted digital data are stored in the storage database provided in the server or remote server or cloud server, and wherein the communication module is selected from a group consisting of a wired network, wireless network, short range communication, Bluetooth communication and near field communication device.

3. The system according to claim 1, wherein the digital writing apparatus further comprises a memory for storing the captured and converted digital data.

4. The system according to claim 1, wherein the tagged writing surface is a prescription sheet, and wherein the prescription sheet comprises pre-set, customized static information, and dynamic information, and wherein the pre-set, customized static information comprises hospital information, doctor information and tag information, and wherein the dynamic information comprises patient's name, patient's age, patient's disease information and suggested treatment for the diseases, and wherein the digital writing apparatus is configured to write dynamic information on the prescription sheet, and wherein the tag on the prescription sheet is a unique identification code or RFID code or QR code for a patient, and wherein the tag is provided to retrieve and digitize information written on the prescription sheet.

5. The system according to claim 1, wherein the computing device is configured to connect to the storage database and retrieve healthcare informatics data related to a plurality of user accounts, and wherein the user accounts are created and configured in the computing device.

6. The system according to claim 1, wherein the storage database is configured to connect to the computing device and deliver contextual healthcare informatics data pertaining to a plurality of user accounts configured to the user device.

7. The system according to claim 1, wherein the computing device is selected from a group consisting of a PC, laptop, palm computer, mini-computer, portable computing device, ipad, ipod, and smart phone.

8. A computer implemented method comprising instructions stored on a non-transitory computer readable storage medium and executed on a computing device provided with a hardware processor and a memory for storing and delivering healthcare informatics data, the method comprising steps of:
creating a prescription sheet comprising preset customized static information, dynamic information and a tag, and wherein the tag is associated/configured with a unique digital identity;
writing dynamic information on the prescription sheet with a digital writing apparatus, wherein the digital writing apparatus is a writing instrument, and wherein the writing instrument comprises an imaging device and a memory, and wherein the imaging device is configured to capture and convert any data physically written on the prescription sheet with the writing instrument into a digital data, and wherein the digital writing apparatus is configured to store the digitized information in the memory;
transmitting the digital data to a server or a remote server or cloud server through a communication module provided in the digital writing apparatus for storage and retrieval of the digitized information, and wherein the transmitted digital data are stored in the storage database provided in the server or remote server or cloud server, and wherein the communication module is selected from a group consisting of a wired network, wireless network, short range communication Bluetooth communication and near field communication device;
storing the dynamic information written on the prescription sheet in the storage database;
tagging the information stored in the storage database with the same tagging information comprised in the prescription sheet; and,
delivering healthcare informatics data related to a particular tag to a plurality of user devices, and wherein the plurality of user devices are configured to receive the healthcare informatics data.

9. The method according to claim 8, wherein the pre-set, customized static information comprises hospital information, doctor information and tag information, and wherein the dynamic information comprises patient's name, patient's age, patient's disease information and suggested treatment for the diseases, wherein the digital writing apparatus is configured to write dynamic information on the prescription sheet, and wherein the tag on the prescription sheet is a unique identification code or RFID code or QR code for a patient, and wherein the tag is provided to retrieve and digitize information written on the prescription sheet.

10. The method according to claim 8, further comprises connecting the computing device to the storage database to retrieve healthcare informatics data related to a plurality of user accounts, and wherein the user accounts are created and configured in the computing device.

11. The method according to claim 8, further comprises connecting the storage database to the computing device to deliver contextual healthcare informatics data related to a plurality of user accounts configured to the user device.

12. The system according to claim 1, wherein the computing device is selected from a group consisting of a PC, laptop, palm computer, mini-computer, portable computing device, computing tablet, iPad, iPod, and smart phone.
